(19) United States
(12) Patent Application Publication
(10) Pub. No.: US 2019/0175107 A1
(43) Pub. Date: Jun. 13, 2019

(54) MULTI-MODAL SENSING ARRAY

(71) Applicant: INTERLINK ELECTRONICS, INC., Westlake Village, CA (US)

(72) Inventors: Chee Wai LU, Singapore (SG); Cheng Seong LEE, Singapore (SG); Wai Jye CHAN, Singapore (SG); Kok Keong LAW, Singapore (SG)

(73) Assignee: INTERLINK ELECTRONICS, INC., Westlake Village, CA (US)

(21) Appl. No.: 16/315,593

(22) PCT Filed: Jul. 10, 2017
(86) PCT No.: PCT/US17/41339

§ 371 Date: Jan. 4, 2019

Related U.S. Application Data
(60) Provisional application No. 62/360,016, filed on Jul. 8, 2016.

(51) Int. Cl. A61B 5/00; A61B 5/103; A61B 5/0205; A43B 3/00; A63B 24/00

(52) U.S. Cl. CPC A61B 5/6807; A61B 5/7455; A61B 5/1038; A61B 5/0205; A61B 5/0022; A61B 5/002; A61B 5/1112; A63B 24/0062; A61B 2560/0257; A61B 2562/0219; A61B 2562/029; A61B 2562/0223; A43B 3/0005

(57) ABSTRACT

An insole system includes a cushion layer configured to contact a human foot within an article of footwear and a sensing layer coupled to the cushion layer. The sensing layer may include a first sensing element and a second sensing element. The first sensing element and the second sensing element are one of: a force sensing element, a strain sensing element, or an environmental sensing element, wherein the first sensing element and the second sensing element are of a different type of sensing element. The insole system may also include a communications interface configured to couple the sensing layer with a host controller.

### FIELD

The embodiments discussed herein are related to a multi-modal sensing array.

### BACKGROUND

The internet of things (IoT) is a network of physical devices, vehicles, buildings and other items—embedded with electronics, software, sensors, actuators, and network connectivity that enable these objects to collect and exchange data, often without user input. IoT devices are sometimes referred to as smart devices. Lately, some advances have been made in developing smart insoles for use in shoes. Conventional smart insole solutions, however, rely primarily on force and/or pressure mapping using force sensing elements. Using force sensing elements alone may present various limitations to smart insoles.

The subject matter claimed herein is not limited to embodiments that solve any disadvantages or that operate only in environments such as those described above. Rather, this background is only provided to illustrate one example technology area where at least one embodiment described herein may be practiced.

### BRIEF DESCRIPTION OF THE DRAWINGS

Example embodiments will be described and explained with additional specificity and detail through the use of the accompanying drawings in which:

FIG. 1 illustrates an arrangement of an example system for sensing various characteristics relating to a human foot and/or a shoe.

FIG. 2 illustrates an example system that includes two sensing arrays.

FIG. 3 illustrates an example computational processing flow for determining various functions including but not limited to foot force/pressure mapping and biomechanics such as foot flexing characteristics, such as during walking or running motion.

FIG. 4 illustrates an example of block diagram for system implementation with an embedded host controller.

FIG. 5 illustrates an example of a block diagram for an alternative system implementation with an embedded host controller.

FIG. 6 illustrates an example implementation of an insole system, where a smart sensing array is connected to an embedded host controller.

FIG. 7 illustrates an example of a graphical user interface (GUI) for parameter monitoring such as foot/pressure mapping based on measurement data from force sensing elements.

FIG. 8 illustrates a block diagram of an example computer system related to a multi-modal array.

### DESCRIPTION OF EMBODIMENTS

Smart insoles are often used for force and/or pressure mapping of a human foot. Conventional smart insole solutions often rely on force and/or pressure mapping using force sensing elements, which typically are integrated within the smart insole as a force sensing layer. Under conventional techniques, the force sensing layer size and geometry must exactly match the shoe size and type, otherwise the force sensing elements may not provide accurate readings. Further, force sensing elements are often sensitive to environmental conditions including temperature and humidity, which may change dramatically based on various activities of a user (such as running). Using force sensing elements alone may present various limitations to smart insoles. For example, a human foot in motion may produce forces in many different directions, which may be difficult to accurately measure in a consistent manner. Further, it may be difficult to measure motion characteristics using force sensing elements alone. For example, while in motion due to external forces (e.g., movement of the user's foot), the insole may experience bending or flexing. Conventional systems may not be able to distinguish or separate these bending or flexing forces from other types of forces.

Aspects of the present disclosure address these and other shortcomings by providing a system (such as a smart insole or a system that may be positioned inside a shoe) for force/pressure mapping and motion measurement within a smart insole, foot pressure monitoring, shoe customization, and other related applications. The system may include one or more environmental sensing elements and one or more strain sensing elements. The one or more environmental sensing elements may include one or more force sensing elements. The one or more strain sensing elements may include two-dimensional strain sensing elements.

In at least one embodiment, an insole may include one or more force sensing elements. The one or more force sensing elements may be configured to detect and/or measure foot force and/or pressure distribution across some or all of a surface of the insole. The insole may include one or more strain sensing elements. The one or more strain sensing elements may be configured to detect and/or measure bending and/or flexing of the insole. The insole may include one or more environmental sensing elements. The one or more environmental sensing elements may be configured to detect and/or measure environmental parameters including temperature and humidity. At least some of the environmental parameters may contribute to noise in the system. The environmental parameters may be accounted for and/or mathematically reduced, minimized or ignored to reduce the noise in the system. Temperature or humidity may contribute toward reduction or modulation of the sensing element output. By measuring the temperature or humidity, self-compensation algorithms can be implemented. The integration of more than one type of sensing elements in combination with signal processing algorithms may provide a robust force and pressure mapping and motion measurement solution.

In at least one embodiment, a system may include a physical stack-up topology that may include a top insole layer, an interposer, one or more force sensing elements, one or more strain sensing elements, one or more environmental sensing elements, and a bottom insole layer. The interposer may include an electrical interface routing between one socket or a connection to another. For example, the interposer may connect any of the one or more force sensing elements, the one or more strain sensing elements, or the one or more environmental sensing elements to a host controller.

In at least one embodiment, an insole system includes a cushion layer configured to contact a human foot within an article of footwear and a sensing layer coupled to the cushion layer. The sensing layer may include a first sensing element and a second sensing element. The first sensing element and the second sensing element are one of: a force sensing element, a strain sensing element, or an environmental sensing element, wherein the first sensing element and the second sensing element are of a different type of sensing element. The insole system may also include a communications interface configured to couple the sensing layer with a host controller.

In at least one embodiment, a system may include a physical arrangement of at least two separate sensing systems. The two separate sensing systems may include a toe area/zone system and a heel area/zone system. For example, the toe area/zone system may be sized and configured to be placed in a toe area/zone of a shoe. Similarly, the heel area/zone system may be sized and configured to be placed in a heel area/zone of a shoe. The two separate sensing systems may enable various configurations for different shoes, sizes, or types, etc. using the same two separate sensing systems. Thus, the same two separate sensing systems may be used to accurately provide force/pressure mapping and motion measurement in different shoes. The two separate sensing systems may be communicatively and/or electrically connected to each other. The two separate sensing systems may be communicatively and/or electrically connected to a host controller.

In at least one embodiment, a system may include at least two sensing layers. Each sensing layer may include one or more force sensing elements which may provide dynamic insole force/pressure detection and measurement within each sensing layer. Each sensing layer may also include one or more strain sensing elements and one or more environmental sensing elements.

In at least one embodiment, a system may include multiple force sensing elements. Each force sensing element may be individually customized for optimal dynamic force/pressure characteristics including but not limited to force/pressure range, rise time, fall time, etc. Each force sensing element may be assigned a specific location within a shoe, location on an insole, and/or a position on a foot. Each force sensing element may be individually customized to measure dynamic force/pressure characteristics based on the respective location within a shoe, location on an insole or position on the foot. The system may also include multiple strain sensing elements, which may provide dynamic insole bending/flexing detection. The system may also include multiple environmental sensing elements which may provide dynamic environmental parameter measurement.

In at least one embodiment, a system may include a processor configured to execute computational processing of dynamic force detection and measurement data from force sensing elements. The processor may use the dynamic force detection and measurement data, for example, to determine a foot force/pressure map across some or all of an insole surface or along the bottom of a user's foot. The processor may also be configured to execute computational processing of dynamic strain detection and measurement data from strain sensing elements. The processor may use the dynamic strain detection and measurement data to determine foot flexing characteristics. The processor may also be configured to execute computational processing of dynamic environmental sensing data received from one or more environmental sensing elements. The processor may also use the environmental sensing data to achieve dynamic environmental compensation of force sensing elements and the strain sensing elements.

In at least one embodiment, the processor may scan the sensing elements (e.g., the force sensing elements, strain sensing elements, and the environmental sensing elements). The processor may scan the sensing elements periodically. In at least one embodiment, the processor may use a variable scanning rate for at least some of the sensing elements, which may provide a benefit of optimal data resolution and/or power consumption. For example, some areas of a user's foot may move more frequently, or may experience a greater rate of change in force or pressure as compared to other areas of the user's foot. These areas may be scanned more frequently for higher data resolution. Those areas with lesser rate of change in force or pressure may be scanned less frequently, which may reduce power consumption of the system.

Systems and method described herein may be used in myriad applications, such as with shoes, insoles, smart sensing mats, flooring, recreational equipment, or other gym or exercise related applications.

Embodiments of the present disclosure are further described with reference to the accompanying drawings.

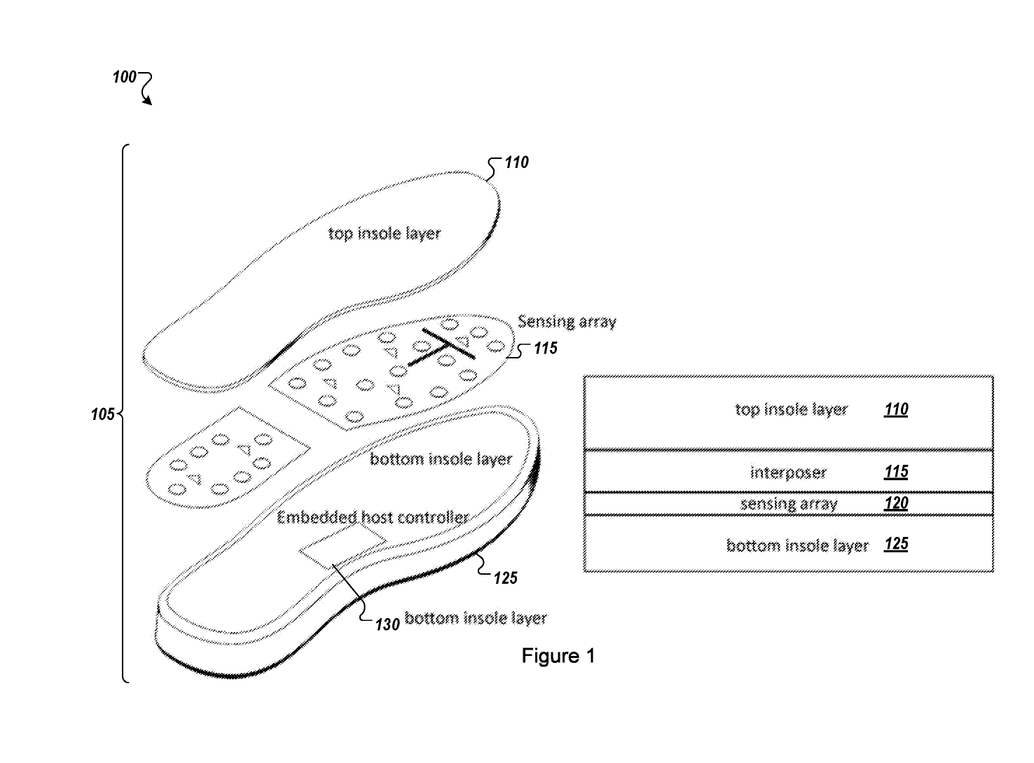

FIG. 1 illustrates an arrangement of an example system 100 for sensing various characteristics relating to a human foot and/or a shoe. The system may be configured to measure force, strain, and other environmental characteristics exerted by a foot or a shoe. The system 100 may include an insole 105 that may be removably inserted within a shoe. Alternatively, the insole 105 may be embedded within or attached to a shoe. As illustrated, the insole 105 may include a top insole layer 110, an interposer 115, one or more sensing elements 120 coupled to the interposer 115, and a bottom insole layer 125.

The top insole layer 110 may be shaped to fit within a shoe, boot, sandal, or any other type of footwear. The top insole layer 110 may be formed from any material or combination of materials. Similarly, the bottom insole layer 125 may be formed from any material or combination of materials. The material may include a porous material, foam material, plastic material, or any other natural or synthetic material. The top insole layer 110 may be composed of different material or materials as the bottom insole layer 125. The bottom insole layer 125 may be formed from a stiffer material or an aggregate stiffness of the bottom insole layer 125 may be stiffer than the top insole layer 110. In at least one embodiment, the bottom insole layer 125 may provide a rigid/stable base for the interposer 115 and/or the sensing elements 120. The top insole layer 110 may be attached to the interposer 115, such as by being bonded (e.g., glued, welded, sewed, etc.) to the interposer 115. In at least one embodiment, the bottom insole layer 125 may be formed from a resilient material configured to withstand repeated impact with a hard surface (e.g., concrete). In at least one embodiment, the bottom insole layer 125 may include or be part of a sole of a shoe.

The interposer 115 may be connected to an external circuit board as part of an external host controller. The interposer 115 may also include or be part of any type of circuit board. The circuit board may be formed from any material. The circuit board may be rigid or flexible.

One or more sensing elements 120 may be coupled to the interposer 115. The one or more sensing elements 120 may be referred to as a sensing array. The one or more sensing elements 120 may include one or more force sensing elements, one or more strain sensing elements, and/or one or more environmental sensing elements. The one or more strain sensing elements may include one or more two-dimensional strain sensing elements. The sensing elements 120 may be spatially distributed on the interposer 115. One or more of the sensing elements 120 may be a discrete part that is coupled to the interposer 115. Alternatively, one or more of the sensing elements 120 may be directly formed, etched, deposited, or printed etc. onto the interposer 115. For example, a sensing element 120 may be printed on a flexible circuit board (i.e., flex).

As illustrated, the system 100 may include two sensing arrays 120. Each sensing array 120 may include one or more force sensing elements which may provide dynamic insole force/pressure detection and measurement within each sensing layer. Each sensing array may also include one or more strain sensing elements and one or more environmental sensing elements. As discussed above, each sensing array may include a toe area/zone array and a heel area/zone array.

The system 100 may also include an embedded host controller 130. The host controller 130 may include circuitry configured to receive data from the sensing elements 120. The host controller 130 may include a memory to store the data and a processor to execute operations. The embedded host controller 130 may be wirelessly connected to a client device (not illustrated) via a communication link. In at least one embodiment, the host controller 130 is coupled to the client device via a wired communication link. The communication link may provide any form of wired or wireless communication capability between the insole 105 and any other device. In some embodiments, the communication link may include a radio frequency (RF) antenna. By way of example and not limitation, the communication link may be configured to provide, via wireless mechanisms, LAN connectivity, Bluetooth connectivity, Wi-Fi connectivity, NFC connectivity, M2M connectivity, D2D connectivity, GSM connectivity, 3G connectivity, 4G connectivity, LTE connectivity, any other suitable communication capability, or any suitable combination thereof. The insole 105 may include any number of communication links.

The host controller 130 may perform various analyses based on data received from the sensing elements 120 (and from any other sensors, as described herein). For example, the host controller 130 may generate a force and/or pressure map of a human foot. The force and/or pressure map may be an instantaneous (or nearly instantaneous) snapshot of a current state of the human foot. The force and/or pressure map may also include data over time and the map may represent average, median, or other values. The map may be used to determine a level of pronation (e.g., overpronation, underpronation, supination). The map may be viewable as a “heat map” which may show force or pressure ranges in different colors. In at least one embodiment, the host controller 130 may send the sensor data to another device (e.g., a server, a client device) for processing. The host controller 130 may also send the sensor data to another portable or wearable device such as smartphone or smartwatch.

The insole 105 may include any number of sensors that may or may not be part of the sensing array. The sensor may represent any hardware or software sensor capable to detect any characteristic of or near the insole (such as data indicative of motion or environment), including but not limited to an accelerometer, gyroscope, altimeter, global positioning system (GPS), pedometer, magnetometer, a thermometer, a humidity sensor, a barometric pressure sensor, a GPS receiver, any other sensor that may detect motion, environmental, or human state, or any combination thereof. Any motion detected by the sensor may be referred to as a motion characteristic. The sensor may detect various motion patterns that may be associated with a particular movement of a human. The sensor may include any suitable system, apparatus, device, or routine capable of detecting or determining one or more of the following: tilt, shake, rotation, swing, and any other motion. For example, the sensor may detect that the insole is periodically moving in a circular manner that is indicative of a tracked individual taking steps (e.g., walking, running). In some embodiments, the sensor may be configured to detect or determine a location of a particular tracked individual. For example, the sensor may include a GPS receiver, a Wi-Fi signal detector, a mobile phone communication network signal detector, a Bluetooth beacon detector, an Internet Protocol (IP) address detector or any other system, apparatus, device, or module that may detect or determine a location of the particular tracked individual. The location may include one or more labels or designations (e.g., home, work, gym). In some embodiments, the sensor may be an integrated sensor that includes two or more different sensors integrated together. For example, the sensor may be an integrated sensor that combines a three-dimensional (3D) accelerometer, a 3D gyroscope, and a 3D magnetometer.

The insole 105 may also include any number of activity trackers. An activity tracker may represent any hardware or software sensor or device that may be used to detect characteristics (or data indicative of the characteristics) of a tracked individual who is using the insole 105, including but not limited to, a heart rate monitor, a blood pressure monitor, thermometer, moisture sensor, respiration sensor, electrodermal activity sensor, sleep sensor, etc. The activity tracker may be used to identify characteristics of the tracked individual who is using the insole 105. In some embodiments, the heart rate monitor may be configured to measure or determine heart rate or indicators of heart rate. For example, the heart rate monitor may include one or more sensors (e.g., a photoresistor or a photodiode or the like) configured to detect a pulse, a skin temperature, etc. of a monitored tracked individual.

In these or other embodiments, the activity tracker may include a heart rate monitor may include one or more systems, apparatuses, devices, or modules configured to determine the heart rate based on the detected indicators. In some embodiments, an occurrence in a life of the particular tracked individual may include a heart rate of the particular tracked individual, a heart rate maintained by the particular tracked individual for a particular amount of time, a heart rate recovery time, etc., which may be determined by the host controller 130 (or by an external computing device) based on data received from one or more heart rate monitors or from other activity trackers or sensors.

The insole 105 may also include any number of haptic feedback devices that may provide any type of haptic feedback to the user.

The insole 105 may include more or fewer features. For example, the insole 105 may not include a top insole layer 110 and the sensing elements 120 may be disposed such that an outward facing surface of each sensing element 120 may be substantially coplanar with a foot-facing surface of the interposer 115. In such an embodiment, the interposer 115 may be formed from a material that may provide some degree of comfort to a human foot. The interposer 115, for example, may be formed from a pliable material. In at least one embodiment, the interposer 115 may include one or more recesses in which the sensing elements 120 may be affixed. In at least some embodiments, one or both of the top insole layer and the bottom insole layer may be referred to as a cushion layer.

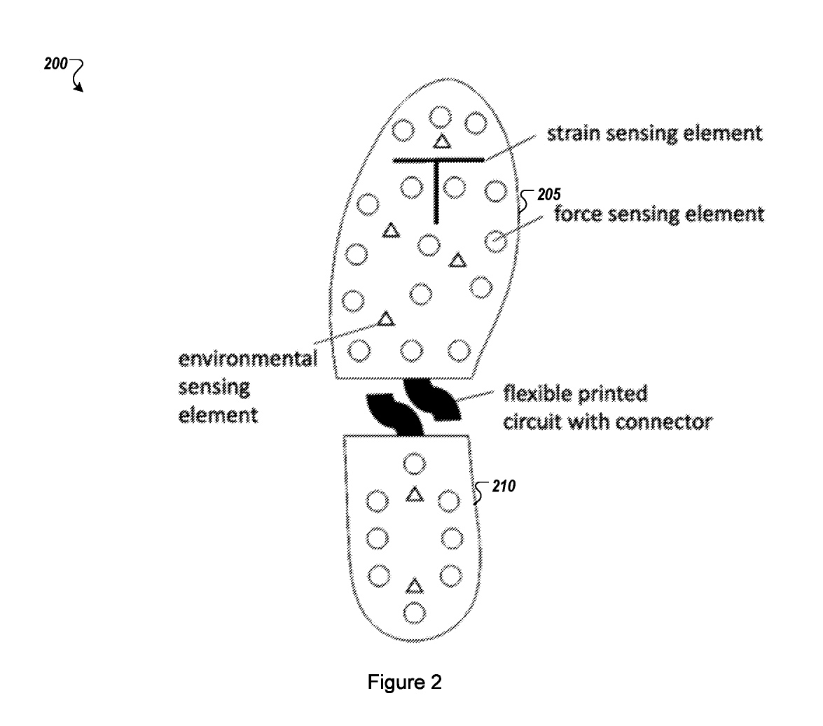

FIG. 2 illustrates an example system 200 that includes two sensing arrays. A first sensing array 205 may be shaped to fit in a first portion of a shoe (e.g., a portion near where a user would place their toes) and a second sensing array 210 may be shaped to fit in a second portion of the shoe (e.g., a portion near where a user would place their heel). Each of the first sensing array 205 and the second sensing array 210 may include any number of sensing elements. As illustrated, the first sensing array 205 includes a strain sensing element, multiple force sensing elements and multiple environmental sensing elements. As illustrated, the second sensing array 210 includes multiple force sensing elements and multiple environmental sensing elements. The first sensing array 205 and the second sensing array 210 may be independently communicatively and/or electronically coupled to a host controller (e.g., the host controller of FIG. 1) via a connector interface. In at least one embodiment, the first sensing array 205 and the second sensing array 210 are communicatively and/or electronically coupled to each other via respective connector interfaces, such as via a flexible printed circuit with a connector. In such an embodiment, either the first sensing array 205 or the second sensing array 210 may be communicatively and/or electronically coupled to the host controller via a connector interface.

The arrangement of two sensing arrays enables reconfiguration of shoe length or size. Additionally, force sensing elements may be located in regions where higher levels of force/pressure may be applied by the foot. Strain sensing elements (e.g., two dimensional strain sensing elements) may be located in regions where higher levels of bending/flexing may be applied by the foot. Environmental sensing elements may be located in regions where there may be minimal levels of force/pressure or bending/flexing but subjected to equivalent environmental parameters as the force sensing and two-dimensional strain sensing elements.

In an example, the system 200 may be used for dynamic motion monitoring in physiotherapy where the goal would be for the user to be able to walk correctly after medical treatment. In another example, the system 200 may be used for dynamic motion monitoring in sports (e.g., track and field) where the user would be able to improve running performance based on the dynamic motion monitoring both in real time and over time. In some embodiments, haptic devices that may provide haptic feedback may be incorporated into the system to stimulate correct behavior.

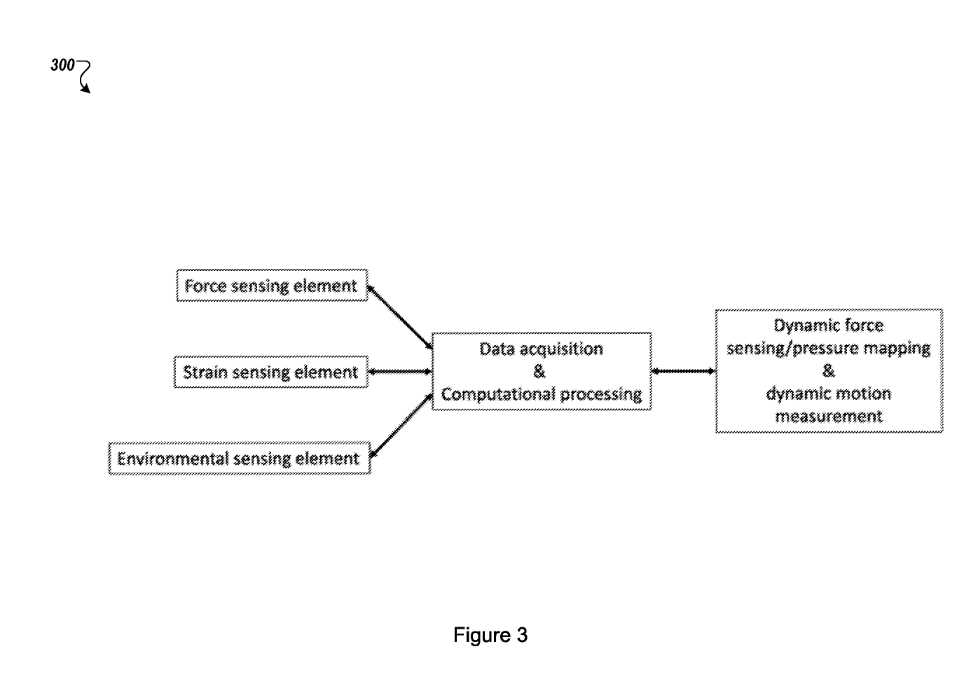

FIG. 3 illustrates an example computational processing flow 300 for determining various functions including but not limited to foot force/pressure mapping and biomechanics such as foot flexing characteristics, such as during walking or running motion. These functions may be obtained by analysis of measurement data from force sensing elements, dimensional strain sensing elements and environmental sensing elements or other sensors or activity trackers.

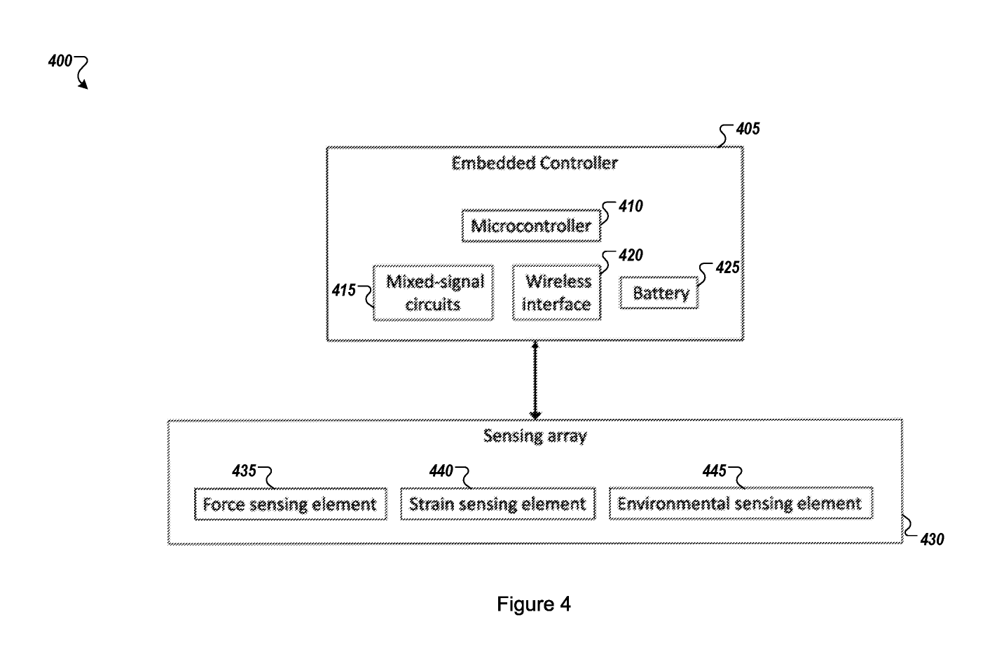

FIG. 4 illustrates an example of block diagram 400 for system implementation with an embedded host controller 405, which may include a microcontroller 410, one or more mixed-signal circuits 415, and peripheral electronics components including wireless data interface 420 and battery 425. As described, the embedded host controller 405 may be communicatively and/or electronically coupled to one or more sensing arrays 430 that may include one or more force sensing element 435, strain sensing element 440, and/or environmental sensing element 445.

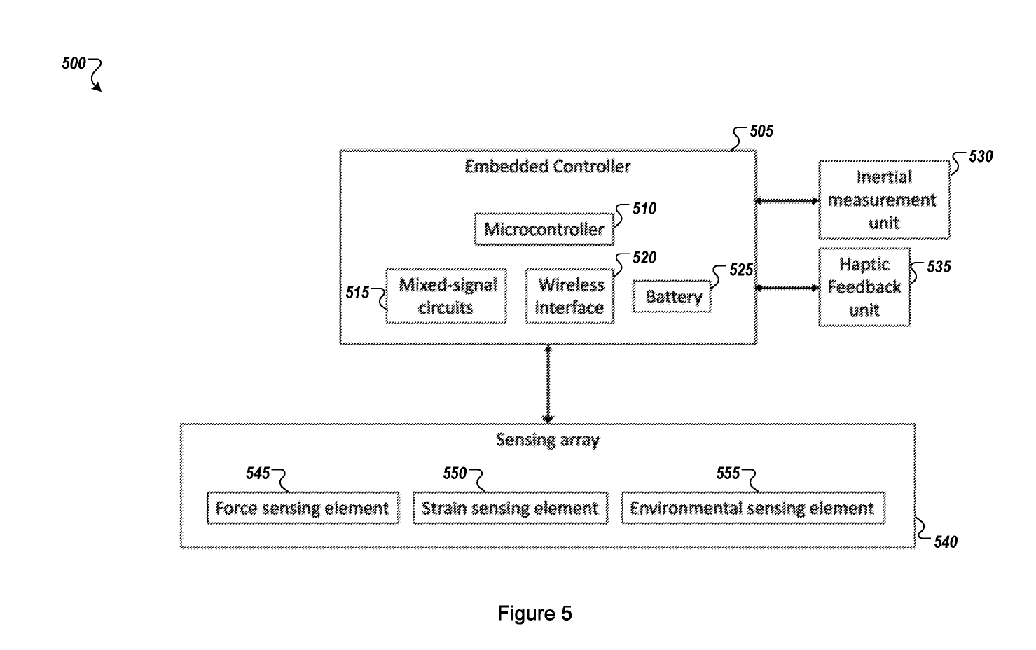

FIG. 5 illustrates an example of a block diagram 500 for an alternative system implementation with an embedded host controller 505, which may include a microcontroller 510, mixed-signal circuits 515 and peripheral electronics components including wireless data interface 520, battery 525, together with an inertial measurement unit 530 including but not limited to an accelerometer and gyroscope. The system may also include a haptic feedback unit 535 that may drive haptic feedback to the system 500. For example, the embedded controller 505 may receive sensor data from a sensing array 540. The sensing array 540 that may include one or more force sensing element 545, strain sensing element 550, and/or environmental sensing element 555. Based on the sensor data, the embedded controller 505 may generate and send instructions to the haptic feedback unit 535 to produce a haptic response via the system 500 (e.g., as a haptic feedback via an insole that the user may feel in their foot).

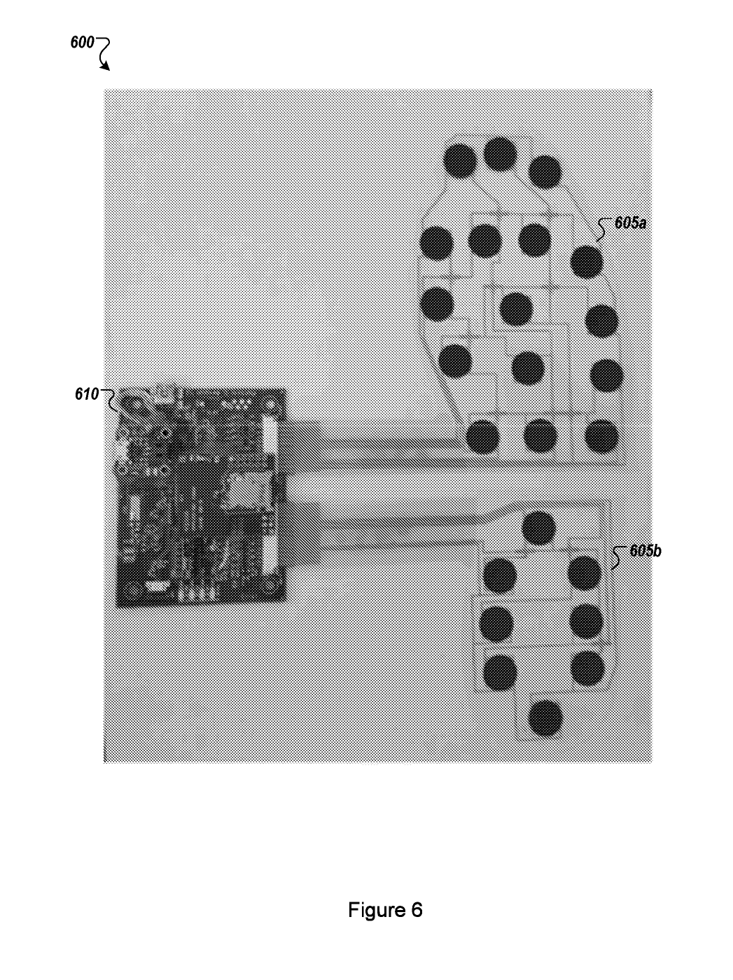

FIG. 6 illustrates an example implementation of an insole system 600, where a smart sensing array 605 is connected to an embedded host controller 610. As illustrated, the insole system 600 includes two sensing arrays 605a, 605b, each being independently coupled to the host controller 610.

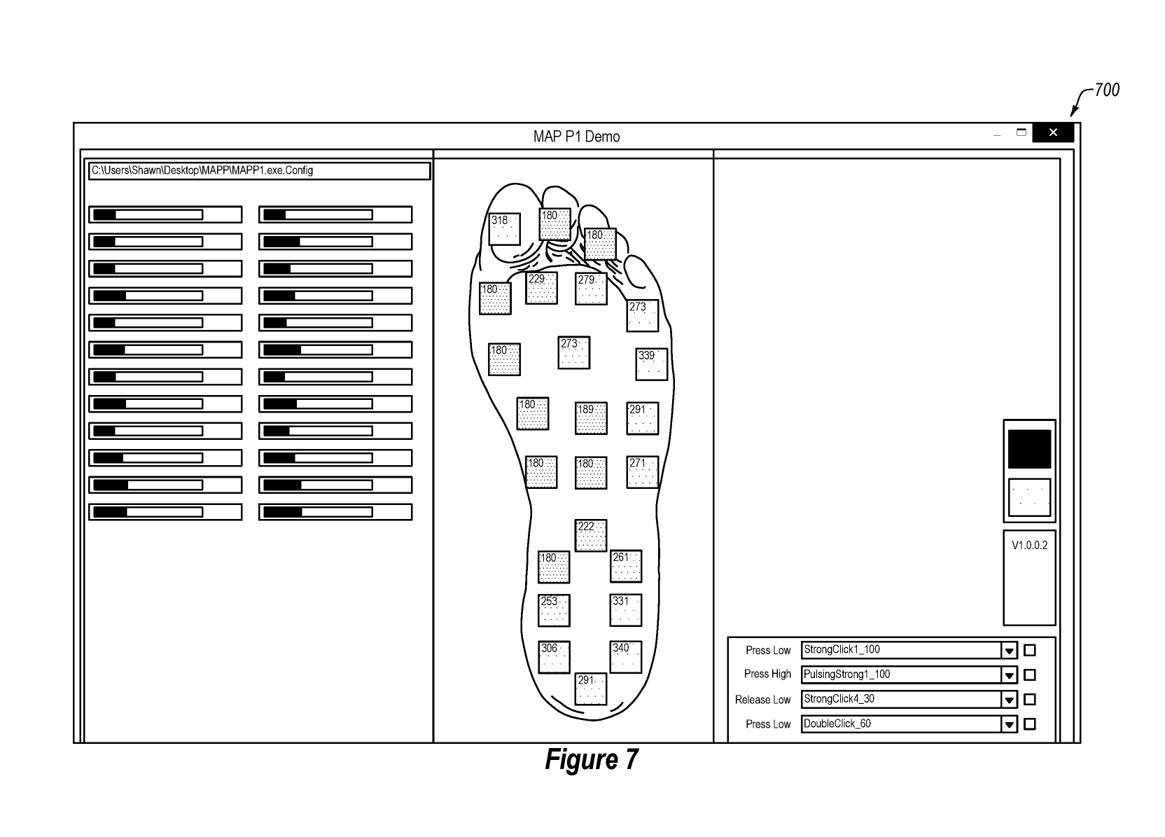

FIG. 7 illustrates an example of a graphical user interface (GUI) 700 for parameter monitoring such as foot/pressure mapping based on measurement data from force sensing elements from any of the systems described.

In at least one embodiment, the GUI 700 may include an outline of a foot (the right foot is shown, but the left foot or both feet may be displayed). The GUI 700 may also include graphical representations of each sensing element (hereafter graphical sensor). A sensed value (e.g., a force value) may be represented in each respective sensing element. As illustrated, each graphical sensor is shaded according to its respective force value. The shading may be color based, pattern based, etc. Also illustrated, each graphical sensor includes a respective force reading. For example, the upper left graphical sensor includes a force reading of “318.” The force readings may be an absolute value (e.g., in Newtons or kg·m/s2) or a relative value.

The GUI 700 may also include other portions that may display data readings. For example, the left portion of the GUI 700 may include data readings in a list format. As illustrated, the bottom right portion of the GUI 700 may highlight an example of haptic feedback settings. The haptic may be provided via a haptic device via the insole that may be felt by the user. Example haptic feedback may include, but are not limited to, a press, a pulse, a shock, a release, all of which may be short, long, or repeated. The haptic feedback may be used to encourage a particular behavior. For example, if a runner favors his heels, the haptic feedback may notify or remind the user when the user is favoring his heels. The user may then adjust his running technique toward his toes and away from his heels.

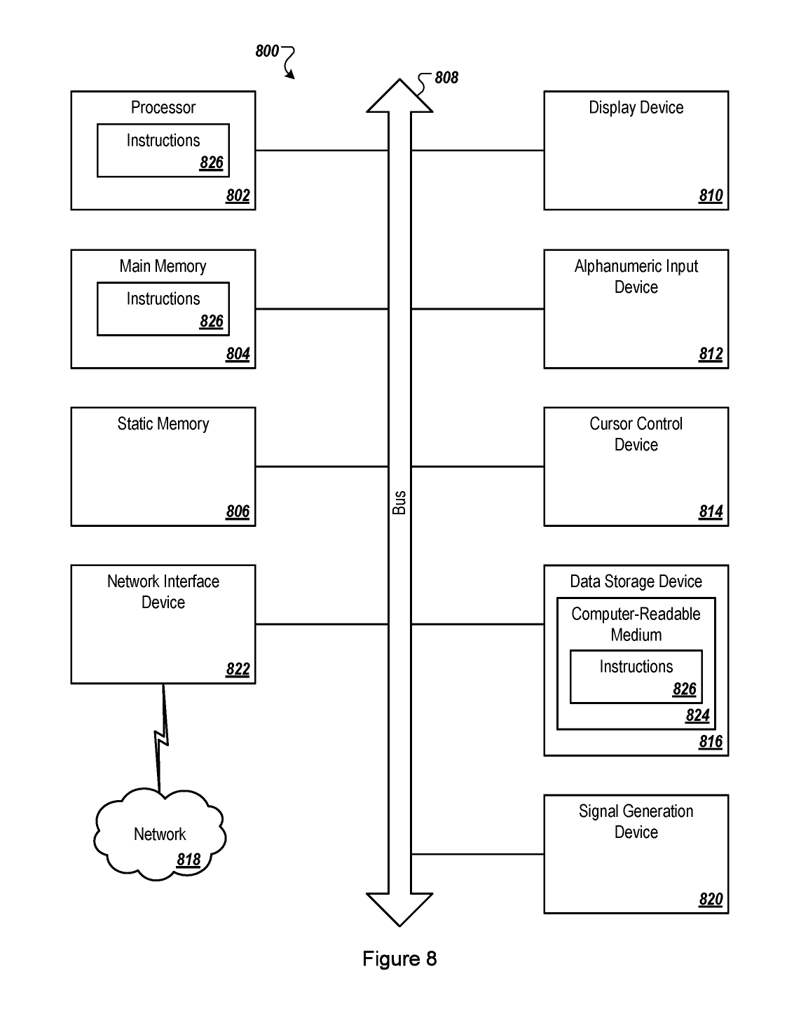

FIG. 8 illustrates a block diagram of an example computer system 800 related to a multi-modal array, according to at least one embodiment of the present disclosure. The host controller described above may be implemented as a computing system such as the example computer system 800. The computer system 800 may be configured to implement one or more operations of the present disclosure.

The computer system 800 executes one or more sets of instructions 826 that cause the machine to perform any one or more of the methods discussed herein. The machine may operate in the capacity of a server or a client machine in client-server network environment, or as a peer machine in a peer-to-peer (or distributed) network environment. The machine may be a personal computer (PC), a tablet PC, a set-top box (STB), a personal digital assistant (PDA), a mobile telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute the sets of instructions 826 to perform any one or more of the methods discussed herein.

The computer system 800 includes a processor 802, a main memory 804 (e.g., read-only memory (ROM), flash memory, dynamic random access memory (DRAM) such as synchronous DRAM (SDRAM) or Rambus DRAM (RDRAM), etc.), a static memory 806 (e.g., flash memory, static random access memory (SRAM), etc.), and a data storage device 816, which communicate with each other via a bus 808.

The processor 802 represents one or more general-purpose processing devices such as a microprocessor, central processing unit, or the like. More particularly, the processor 802 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 802 may also be one or more special-purpose processing devices such as an application specific integrated circuit (ASIC), a field programmable gate array (FPGA), a digital signal processor (DSP), network processor, or the like. The processor 802 is configured to execute instructions for performing the operations and steps discussed herein.

The computer system 800 may further include a network interface device 822 that provides communication with other machines over a network 818, such as a local area network (LAN), an intranet, an extranet, or the Internet. The network interface device 822 may include any number of physical or logical interfaces. The network interface device 822 may include any device, system, component, or collection of components configured to allow or facilitate communication between network components in a network. For example, the network interface device 822 may include, without limitation, a modem, a network card (wireless or wired), an infrared communication device, an optical communication device, a wireless communication device (such as an antenna), and/or chipset (such as a Bluetooth device, an 802.xx device (e.g. Metropolitan Area Network (MAN)), a WiFi device, a WiMax device, cellular communication facilities, etc.), and/or the like. The network interface device 822 may permit data to be exchanged with a network (such as a cellular network, a WiFi network, a MAN, an optical network, etc., to name a few examples) and/or any other devices described in the present disclosure, including remote devices. In at least one embodiment, the network interface device 822 may be logical distinctions on a single physical component, for example, multiple communication streams across a single physical cable or optical signal.

The computer system 800 also may include a display device 810 (e.g., a liquid crystal display (LCD) or a cathode ray tube (CRT)), an alphanumeric input device 812 (e.g., a keyboard), a cursor control device 814 (e.g., a mouse), and a signal generation device 820 (e.g., a speaker).

The data storage device 816 may include a computer-readable storage medium 824 on which is stored the sets of instructions 826 embodying any one or more of the methods or functions described herein. The sets of instructions 826 may also reside, completely or at least partially, within the main memory 804 and/or within the processor 802 during execution thereof by the computer system 800, the main memory 804 and the processor 802 also constituting computer-readable storage media. The sets of instructions 826 may further be transmitted or received over the network 818 via the network interface device 822.

While the example of the computer-readable storage medium 824 is shown as a single medium, the term “computer-readable storage medium” may include a single medium or multiple media (e.g., a centralized or distributed database, and/or associated caches and servers) that store the sets of instructions 826. The term “computer-readable storage medium” may include any medium that is capable of storing, encoding or carrying a set of instructions for execution by the machine and that cause the machine to perform any one or more of the methods of the present disclosure. The term “computer-readable storage medium” may include, but not be limited to, solid-state memories, optical media, and magnetic media.

Modifications, additions, or omissions may be made to the computer system 800 without departing from the scope of the present disclosure. For example, in at least one embodiment, the computer system 800 may include any number of other components that may not be explicitly illustrated or described.

The following examples pertain to further embodiments.

Example 1 includes an insole system includes a bottom layer and a sensing layer coupled to the bottom layer, the sensing layer includes a first sensing element and a second sensing element, where the first sensing element and the second sensing element are one of: a force sensing element, a strain sensing element, or an environmental sensing element, where the first sensing element and the second sensing element are of a different type of sensing element; and a communications interface configured to couple the sensing layer with a host controller.

In Example 2, the subject matter of Example 1 and a top insole layer coupled to the sensing layer.

In Example 3, the subject matter of any one of Examples 1-2, where at least one of the first sensing element and the second sensing element are at least partially embedded in the top insole layer.

In Example 4, the subject matter of any one of Examples 1-3, where the top insole layer is formed from a first material and the bottom layer is formed from a second material.

In Example 5, the subject matter of any one of Examples 1-4, where the communications interface is at least partially embedded in one or both of the first sensing element and the second sensing element.

In Example 6, the subject matter of any one of Examples 1-5, where at least one of the first sensing element and the second sensing element are at least partially embedded in the bottom layer.

In Example 7, the subject matter of any one of Examples 1-6 further including a controller operatively coupled to the sensing layer and to the communications interface.

In Example 8, the subject matter of any one of Examples 1-7, where the controller is configured to perform operations including: receive data indicative of a detected force on the sensing layer; and send the data to an external device via the communications interface.

In Example 9, the subject matter of any one of Examples 1-8, where the controller is configured to generate, based on the data indicative of the detected force on the sensing layer, a force map that indicates a plurality of forces at a plurality of corresponding positions.

In Example 10, the subject matter of any one of Examples 1-9, further including an additional sensor selected from a group of sensors consisting of an accelerometer, gyroscope, altimeter, global positioning system (GPS), pedometer, magnetometer, a thermometer, a humidity sensor, a barometric pressure sensor, a GPS receiver, a Wi-Fi signal detector, a mobile phone communication network signal detector, a Bluetooth beacon detector, or an Internet Protocol (IP) address detector.

In Example 11, the subject matter of any one of Examples 1-10 further including an activity tracker that is configured to data indicative of characteristics of a tracked individual who is using the insole system.

In Example 12, the subject matter of any one of Examples 1-11 further including a haptic feedback device that is configured to provide haptic feedback to a user based at least in part on force detected by the sensing layer.

Example 13 is an insole system, including a cushion layer configured to contact a human foot within an article of footwear; a sensing layer coupled to the cushion layer, the sensing layer including a first sensing element and a second sensing element, where the first sensing element and the second sensing element are one of: a force sensing element, a strain sensing element, or an environmental sensing element, where the first sensing element and the second sensing element are of a different type of sensing element; and a communications interface configured to couple the sensing layer with a host controller.

In Example 14, the subject matter of Examples 13, where at least one of the first sensing element and the second sensing element are at least partially embedded in the cushion layer.

In Example 15, the subject matter of any one of Examples 1-14, where the cushion layer includes a top layer and a bottom layer.

In Example 16, the subject matter of any one of Examples 1-15 further including one or more processors being configured to perform operations including: detect a force on the sensing layer by at least one of the first sensing element or the second sensing element; measure at least one of a magnitude and a direction of the detected force; compute force sensing data parameters based on the measuring; determine an output function based on the force sensing data parameters; and send the output function for display in a graphical user interface.

In Example 17, the subject matter of any one of Examples 1-16 further including an additional sensor configured to receive environmental data, the one or more processors being configured to perform further operations including identify environmental data from the additional sensor; and adjust the output function based on the environmental data.

Example 18 is a method including detecting a force on an insole by a sensing element of the insole; measuring at least one of a magnitude and a direction of the detected force; computing force sensing data parameters based on the measuring; determining an output function based on the force sensing data parameters; and sending the output function for display in a graphical user interface.

In Example 19, the subject matter of Example 18, where sending the output function for display in the graphical user interface comprises providing the output function via the graphical user interface.

In Example 20, the subject matter of any one of Examples 18-19 further including identifying environmental data from an additional sensor; and adjusting the output function based on the environmental data.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, etc.) of the computing system. In at least one embodiment, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated. In the present disclosure, a “computing entity” may be any computing system as previously defined in the present disclosure, or any module or combination of modulates running on a computing system.

Terms used in the present disclosure and especially in the appended claims (e.g., bodies of the appended claims) are generally intended as “open” terms (e.g., the term “including” may be interpreted as “including, but not limited to,” the term “having” may be interpreted as “having at least,” the term “includes” may be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases may not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” may be interpreted to mean “at least one” or “one or more”); the same holds true for the use of definite articles used to introduce claim recitations.

In addition, even if a specific number of an introduced claim recitation is explicitly recited, those skilled in the art will recognize that such recitation may be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, may be understood to contemplate the possibilities of including one of the terms, either of the terms, or both terms. For example, the phrase “A or B” may be understood to include the possibilities of “A” or “B” or “A and B.”

All examples and conditional language recited in the present disclosure are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, various changes, substitutions, and alterations may be made hereto without departing from the spirit and scope of the present disclosure.

What is claimed is:

1. An insole system, comprising:
a bottom layer;
a sensing layer coupled to the bottom layer, the sensing layer comprising a first sensing element and a second sensing element, wherein the first sensing element and the second sensing element are one of: a force sensing element, a strain sensing element, or an environmental sensing element, wherein the first sensing element and the second sensing element are of a different type of sensing element; and
a communications interface configured to couple the sensing layer with a host controller.

2. The insole system of claim 1 further comprising a top insole layer coupled to the sensing layer.

3. The insole system of claim 2, wherein at least one of the first sensing element and the second sensing element are at least partially embedded in the top insole layer.

4. The insole system of claim 2, wherein the top insole layer is formed from a first material and the bottom layer is formed from a second material.

5. The insole system of claim 2, wherein the communications interface is at least partially embedded in one or both of the first sensing element and the second sensing element.

6. The insole system of claim 1, wherein at least one of the first sensing element and the second sensing element are at least partially embedded in the bottom layer.

7. The insole system of claim 1 further comprising a controller operatively coupled to the sensing layer and to the communications interface.

8. The insole system of claim 7, wherein the controller is configured to perform operations comprising:
receive data indicative of a detected force on the sensing layer; and
send the data to an external device via the communications interface.

9. The insole system of claim 8, wherein the controller is configured to generate, based on the data indicative of the detected force on the sensing layer, a force map that indicates a plurality of forces at a plurality of corresponding positions.

10. The insole system of claim 1 further comprising an additional sensor selected from a group of sensors consisting of an accelerometer, gyroscope, altimeter, global positioning system (GPS), pedometer, magnetometer, a thermometer, a humidity sensor, a barometric pressure sensor, a GPS receiver, a Wi-Fi signal detector, a mobile phone communication network signal detector, a Bluetooth beacon detector, or an Internet Protocol (IP) address detector.

11. The insole system of claim 1 further comprising an activity tracker that is configured to data indicative of characteristics of a tracked individual who is using the insole system.

12. The insole system of claim 1 further comprising a haptic feedback device that is configured to provide haptic feedback to a user based at least in part on force detected by the sensing layer.

13. An insole system, comprising:
a cushion layer configured to contact a human foot within an article of footwear;
a sensing layer coupled to the cushion layer, the sensing layer comprising a first sensing element and a second sensing element, wherein the first sensing element and the second sensing element are one of: a force sensing element, a strain sensing element, or an environmental sensing element, wherein the first sensing element and the second sensing element are of a different type of sensing element; and
a communications interface configured to couple the sensing layer with a host controller.

14. The insole system of claim 13, wherein at least one of the first sensing element and the second sensing element are at least partially embedded in the cushion layer.

15. The insole system of claim 13, wherein the cushion layer includes a top layer and a bottom layer.

16. The insole system of claim 13 further comprising one or more processors being configured to perform operations comprising:
detect a force on the sensing layer by at least one of the first sensing element or the second sensing element;
measure at least one of a magnitude and a direction of the detected force;
compute force sensing data parameters based on the measuring;
determine an output function based on the force sensing data parameters; and
send the output function for display in a graphical user interface.

17. The insole system of claim 16 further comprising an additional sensor configured to receive environmental data, the one or more processors being configured to perform further operations comprising:
identify environmental data from the additional sensor; and
adjust the output function based on the environmental data.

18. A method comprising:
detecting a force on an insole by a sensing element of the insole;
measuring at least one of a magnitude and a direction of the detected force;
computing force sensing data parameters based on the measuring;
determining an output function based on the force sensing data parameters; and
sending the output function for display in a graphical user interface.

19. The method of claim 18, wherein sending the output function for display in the graphical user interface comprises providing the output function via the graphical user interface.

20. The method of claim 18 further comprising:
identifying environmental data from an additional sensor; and
adjusting the output function based on the environmental data.
